(19) United States
(12) Patent Application Publication
(10) Pub. No.: US 2012/0310357 A1
(43) Pub. Date: Dec. 6, 2012

(54) POROUS TITANIUM

(76) Inventors: Lars Magnus Bjursten, Limhamn (SE); Bengt Mjöberg, Ystad (SE)

(21) Appl. No.: 13/513,897

(22) PCT Filed: Dec. 16, 2010
(86) PCT No.: PCT/SE10/51400

§ 371 Date: Aug. 20, 2012

Related U.S. Application Data
(60) Provisional application No. 61/282,110, filed on Dec. 17, 2009.

(30) Foreign Application Priority Data
Dec. 17, 2009 (SE) 0950972-0

(51) Int. Cl. A61F 2/02

(52) U.S. Cl. 623/17.17

(57) ABSTRACT

The present invention provides a porous block for implantation in the maxillofacial area of a human or animal, wherein the porous block:—comprises titanium metal and/or a titanium alloy;—has a porosity of at least 40%;—is a geometrical structure shaped to fit at least a part of a degraded alveolar process of the human or animal; and—as an intended bone contacting surface intended to be in contact with the bone surface of an implantation site in the maxillofacial area of the human or animal, wherein said bone contacting surface has pores extending through the porous block and wherein at least some of these pores have a pore diameter size of at least 50 μm to ensure bone ingrowth; and wherein—the porous block is a geometrical structure having a width, a height and a length, said intended bone contacting surface being defined by the width and the length, wherein the average value of the width is in the range of 5-10 mm, the average value of the height is in the range of 3-10 mm and the average value of the length is in the range of 5-100 mm.

### FIELD OF THE INVENTION

The present invention relates to a porous titanium and/or titanium alloy block and to the use thereof as an implant in the maxillofacial area of a human or animal subject. Moreover, according to one specific embodiment of the present invention, there is provided a porous block of titanium and/or a titanium alloy having an intended bone contacting surface being porous but with other surfaces being non-porous, such as by an non-permeable surface layer on the otherwise porous block or by means of a separate membrane in a kit or by the provision of a protective film directly on these surfaces making them non-porous.

### BACKGROUND OF THE INVENTION

It is known that operations and wounds in the body often brings about inflammation and/or infections, which is the case also in connection with implantations, especially in connection with bone tissue, e. g. hip joints and dental applications.

When titanium is exposed to air or water, an oxide layer is spontaneously formed. This spontaneously formed oxide layer is 4-10 nm thick and consists predominantly of TiO₂, Ti(IV), with smaller amounts of Ti(III) and Ti(II) present in the oxide.

Titanium (that is titanium metal with a surface layer of titanium oxide) has been reported to reduce inflammation (Overgaard, Danielsen et al. 1998) and also to be less susceptible to infections than other materials (Johansson, Lindgren et al. 1999). There are also reports describing unique properties of titanium due to its chemical interactions with reactive oxygen species (ROS). The catalytic property of titanium has been shown to be related to the titanium oxide on the surface being present on surfaces composed of only titanium oxide (Sahlin 2006 et al). Such a catalytic property is e.g. described in the US patent application No. 2005074602 to Bjursten et al and also in the generation of titanium peroxy compounds (Tengvall, Elwing et al. 1989; Tengvall, Lundstrom et al. 1989) with anti-inflammatory (Larsson, Persson et al. 2004) and bactericidal properties (Tengvall, Hornsten et al. 1990). The above beneficial properties of titanium seems thus to be linked to its chemical interaction with a living tissue environment.

WO00/64504 (Bruce et al) discloses a biocompatible, plastic or essentially non-elastic, porous body, such as a grain, with continuous porosity, the openings of cavities and the passages interconnecting them having a width of 50 μm or more for bone tissue. The term “continuous” is said to mean a porosity which allows bone tissue to grow through the porous body. The porous body may be of titanium.

PCT/SE2007/000984 (Bjursten et al.) describes an implant with anti-inflammatory or antibacterial effects, or both, the implant being intended for implantation in a human or an animal body, the implant comprising at least one porous grain or granule, wherein the at least one porous grain or granule comprises titanium, one or more titanium oxides or titanium alloy and has a titanium oxide layer on its surface; has a mean length from one side to the opposite side, through a geometrical centre, of up to 5 mm; has a mean specific surface area of at least 0.15 m²/g according to the BET method. According to PCT/SE2007/000984, the expression “implant” implies the form of a single piece body, including one grain or granule or an agglomerate of particles and/or grains, bonded together or not. Moreover, according to one specific embodiment according to PCT/SE2007/000984, the implant has a titanium oxide layer on its surface with a substantial thickness of at least 500 nm and is yellowish and/or whitish.

As mentioned, both of the implants described in WO00/64504 and PCT/SE2007/000984 are specified as grains or granules. These grains or granules are intended for use in implant applications, as a group together with similar grains or granules, e.g. for filling a cavity in connection with dental and orthopaedic surgeries. There are however situations where other factors are more important for the clinical success of the procedure. An example of this is when the anatomy of the bone defect makes it difficult to place and retain granulate materials. A possible solution in such cases is to enclose the grains or granules completely or partially using a membrane or a sack. However, there exist application areas where these granule structures are not optimal for use, such as when a dentist or surgeon would like to fixate a structure directly on top of living bone to generate new bone, i.e. where there is no natural occurring or surgically made cavity present.

As an example, there is sometimes a defect in the bone of a human or an animal due to trauma, degenerative disease or loss of mechanical stimulation. In order to be augmented such a defect there is a use for a material that can be shaped to fit into the defect. In some cases the defect is on the outer surface of a bone. This makes it difficult to apply and retain a granulate material disclosed above.

In “Ceramic TiO₂-foams: characterisation of a potential scaffold” Haugen et al. discloses the production of ceramic TiO₂ scaffolds. These scaffolds show a fully open structure having a window size (pore size) of 445 pm (45 ppi foams) and 380 pm for the 60 ppi foams. The mean porosity of all foams are said to be 78%. The scaffolds according to Haugen et al. may be improved extensively for the generation of bone ingrowth, e.g. if used when a dentist or surgeon would like to fixate such a structure directly on top of living bone to generate new bone. This is inter alia due to both the geometrical structure as well as the porosity characteristics of the scaffolds according to Haugen et al.

The present invention aims at solving the different problems disclosed above by the provision of a structure which is optimised for fixation directly in contact with living bone.

### SUMMARY OF THE INVENTION

The object above is solved by a porous block according to the present invention, for implantation in the maxillofacial area of a human or animal, wherein the porous block:
comprises titanium metal and/or a titanium alloy;
has a porosity of at least 40%;
is a geometrical structure shaped to fit at least a part of a degraded alveolar process of the human or animal; and
has an intended bone contacting surface intended to be in contact with the bone surface of an implantation site in the maxillofacial area of the human or animal, wherein said bone contacting surface has pores extending through the porous block and wherein at least some of these pores have a pore diameter size of at least 50 μm to ensure bone ingrowth; wherein
the porous block is a geometrical structure having a width, a height and a length, said intended bone contacting surface being defined by the width and the length, wherein the average value of the width is in the range of 5-10 mm, the average value of the height is in the range of 3-10 mm and the average value of the length is in the range of 5-100 mm; and wherein
the porous block comprises an outer layer of titanium oxide on one or more surface sides or surface portions of the porous block.

The block according to the present invention comprises titanium metal or a titanium alloy. In the case of a titanium alloy, such an alloy may comprise other metals than titanium, such as zirconium, tantalum, hafnium, niobium, aluminium, vanadium, molybdenum, chrome, cobalt, magnesium, iron, gold, silver, copper, mercury, tin and zinc. Specific examples of possible alloys are titanium-6 aluminium-4 vanadium, titanium-6 aluminium-7 niobium, titanium-13 niobium-13 zirconium and titanium-12 molybdenum-6 zirconium-2 iron, of which titanium-6 aluminium-4 vanadium and titanium-6 aluminium-7 niobium are most suitable for dental applications.

According to one specific embodiment of the present invention, the porous block comprises titanium metal and/or titanium alloy in an amount of at least 5 wt % in total, such as at least 25 wt %, at least 50 wt %, at least 75 wt %, at least 95 wt % or at least 99 wt % in total. According to one specific embodiment of the present invention, the porous block according to the present invention comprises substantially only technical pure titanium metal except for an outer, naturally occurring, titanium dioxide layer on the surface of the porous block. This oxide layer on the surface is naturally formed in view of oxidation in normal air. However, according to one specific embodiment of the present invention, the outer layer of titanium oxide on one or more surface sides or surface portions of the porous block is applied by a performed oxidation. In other words, such a titanium oxide layer(s), e.g. titanium dioxide layer, may be actively applied by an oxidation, such as an oxidation in high temperatures and oxidizing conditions.

Some trace amounts of impurities are of course possible in the porous block according to the present invention, however, the porous block according to the present invention shall be seen as a block comprising a titanium metal and/or titanium alloy, optionally having an outer titanium oxide or titanium alloy oxide layer. Therefore, according to one specific embodiment of the present invention, the porous block has some titanium oxide coating to achieve specific goals, especially aesthetic. Thus, a titanium oxide on the non-porous surface is preferable according to some embodiments of the present invention. Such oxidation may be achieved by protecting the rest of the block by cooling or by adding a protective element that allows access of the oxidizing conditions to this surface only. In other embodiments a titanium surface oxide on more than the non-porous surface is desirable. Such oxidation is achieved by a partial oxidation. This can be done by exposing the block to an oxidizing gas, e.g. air at elevated temperature or in a wet-chemical environment with strong oxidants or in an electrochemical process such as anodic oxidation. However, a full oxidation to a block consisting entirely of titanium oxide is not desirable and such a 100% titanium oxide block, such as a block comprising 100% titanium dioxide, is not a part of the scope of the present invention.

Titanium dioxide has advantages. This oxide has a whitish colour which is advantageous for aesthetic reasons in dental applications. However, the inventors have found out that a block entirely consisting of titanium dioxide may be difficult to use in some dental applications. This is due to the fact that titanium dioxide may be difficult to reshape for a dentist. Moreover, such a titanium dioxide block is difficult to drill into, such as to make screw holes, which is explained below. Such drill holes are of importance to easily create to make sure that a block is possible to anchor in dental applications, such as with a titanium screw. The inventors have found out that titanium metal and titanium alloy metal, especially titanium metal, are preferable for drilling operations when creating such screw holes. Moreover, titanium dioxide does not sustain such high stresses which titanium metal or titanium alloy may bear.

Therefore, it is according to the present invention preferable that the block comprises at least 50 wt % titanium metal and/or titanium alloy in total, preferably at least 75 wt %, such as at least 80 wt %, e.g. at least 85%, at least 90 wt %, at least 95 wt %, e.g. at least 99 wt % titanium metal and/or titanium alloy in total.

As may be evident from above, it may according to the present invention be of interest to create a block having both the beneficial properties of titanium metal and titanium dioxide. Such a block according to the present invention comprises titanium metal and/or titanium alloy in its core and has a titanium dioxide layer in its surface, especially in the non-permeable surface thereof. This titanium dioxide layer is provided on at least one portion or side surface of the block. According to one embodiment of the present invention, such at least one outer titanium dioxide layer on at least one surface side or surface portion has a substantial thickness of at least 500 nm making sure that the block has a yellowish and/or whitish appearance. This refers especially to the non-permeable surface or surfaces of the block. Nevertheless, also according to this specific embodiment of the present invention the inner core of the block is made of titanium metal and/or titanium alloy ensuring the properties of being easy to reshape and drill into.

As is explained above, if titanium oxide is of interest to provide on the surface of the block according to the present invention, the inventors have found out that only sides intended to be non-porous are of real interest to be arranged with a titanium oxide coating, such as a titanium dioxide coating. Other sides intended to stay porous may consist of titanium metal or a titanium alloy and still have the intended clinical properties.

The porosity of the block according to the present invention is also an important feature. Without any porosity there would not exist any possibility of bone ingrowth into the blocks. However, the porosity of the blocks according to the present invention has to be provided at the right places of the blocks and not with a fully open structure such as according to Haugen et al. This is due to the fact that connective tissue grows more easily and faster into pores of a block after implantation in comparison to bone tissue, and due to the fact that such an ingrowth therefore depresses bone ingrowth, it has to be controlled and hindered. With a fully open structure, connective tissue grows into the pores from one side and throughout the block, and as such therefore hinders bone ingrowth from the intended side when being used in e.g. dental applications.

Moreover, the shape of the block according to the present invention is also an important technical feature. The blocks according to the present invention are intended for bone generation in the maxillofacial area of a human or animal. Therefore, these blocks have to be shaped to fit at least a part of or an entire alveolar process of a human or animal.

As mentioned, according to state of the art, a possible solution for placing and retaining a granulate material, when the anatomy of the bone defect makes that difficult, is to enclose the granules completely or partially using a membrane or a sack. The present invention is a substantial improvement over such complicated procedures. It is, however, according to the present invention possible to optimise the surface porosity of the block according to the present invention or to apply the block(s) with a membrane to further improve the chances of bone ingrowth into the block(s). Optimal bone ingrowth is not possible to achieve in the maxillofacial area by use of scaffolds according to Haugen et al., which is due to both the shape and the fully open structure of the scaffolds according to Haugen et al. In other words, due to the fact that these scaffolds are not preformed to fit an intended implantation site of the maxillofacial area and due to that these structures do not include a membrane to hinder soft tissue ingrowth, they are not optimal for bone ingrowth in the maxillofacial area.

When there is a defect in the bone of a human or an animal situated on the outer surface of the bone, a porous block according to the present invention can easily be fixed to the bone tissue and provide good bone ingrowth. The shape of the block may be pre-shaped to fit the individual patient's needs by using CAD CAM techniques, but ideally the block should be possible to shape or adjust while the patient is undergoing surgery. At the same time the block must possess enough mechanical strength to sustain the loads that it may become exposed to.

As should be understood from above, the structure of the implant according to the present invention is a block, and not a particle, grain or granule. The block structure according to the present invention can be compared to that of e.g. pumice stone. Other implant types, such as particle, grain or granule implants are intended to be used together with other ones, such as for e.g. filling a cavity, e.g. in a cavity around a prosthesis. These particle, grain or granule implants are compacted in such a cavity. Moreover, these particles, grains or granules are of randomised structure, i.e. the shape of these particles, grains or granules are not designed during the production thereof. The porous block according to the present invention, however, has a designed structure for the intended use thereof. Furthermore, the blocks according to the present invention are not intended to be used as a randomised group together with other blocks, which is the case for the particles, grains or granules, such as when these particles, grains or granules are vibrated in a cavity surrounding a prosthesis. However, a porous block according to the present invention may be used together with another or several blocks according to the invention, such as to form a bridge of blocks, where the bridge is designed to fit the intended implantation site in a suitable way. The distinguishing difference to the previously described particles, grains or granules is that for these no or low mechanical stability is needed or the stability is provided by the interlocking of several particles, grains or granules. The blocks according to present invention, however, are each by themselves providing the needed stability, although there may be situations where a couple of blocks are used in order to best fit the bone defect. This is possible due to the structural difference of a block according to the present invention in comparison to smaller structures, such as the mentioned particles, grains or granules.

### Explanations of Some Important Expressions

The expression “comprises titanium metal and/or a titanium alloy” implies that the block according to the present invention comprises either titanium metal or a titanium alloy, or actually comprises portions consisting of titanium metal and also other portions consisting of a titanium alloy. Moreover, “comprises” should in this case be interpreted to imply that at least a portion of the block consists of titanium metal or a titanium alloy. Preferably, a substantial part of the block consists of titanium metal or a titanium alloy, such as at least more than 50 wt % in total, e.g. more than 75 wt % in total, such as at least 80 wt % in total, at least 85 wt % in total, at least 90 wt % in total, at least 95% in total, e.g. at least 99 wt % in total. As is explained above, according to one embodiment, the block core consists of titanium metal or a titanium alloy and there is provided a titanium oxide layer on the surface, such as a titanium dioxide layer. In the case when, in comparison, only a small amount of titanium metal or titanium alloy is comprised in the block, other components in the block may e.g. be a substantial layer of titanium oxide, such as titanium dioxide, going from the surface towards the core, but also other possible biocompatible materials, such as biocompatible metals, which is not part of a titanium alloy. The latter should, however, in such a case normally exist in small amounts. Nevertheless, at least a part of the block comprises titanium metal or a titanium alloy.

With reference to above, the expression “in total” implies that if both titanium metal and titanium alloy are existing separately in the block, the amount referred to is the total weight amount of both titanium metal and the titanium alloy. A block consisting of both titanium metal and titanium alloy in its core is not preferable. However, one possible embodiment of the present invention, when both titanium metal and titanium alloy may be present, is a block having a non-porous laminated surface layer (film) on one or more sides of the block. In this case, the laminated surface layer may have a composition which is another one than e.g. the titanium metal or titanium alloy of the core of the block. Therefore, according to the present invention both titanium metal and a titanium alloy may be present in/on the block. For example, a block having a core of titanium metal and with a laminated surface layer of titanium alloy provided on at least one side of the block is possible, and vice versa. Such a laminated surface layer film may be provided on sides which are porous but intended to be non-porous. Moreover, according to the present invention, such a laminated surface layer may have been surface treated to get a whitish appearance.

The expression “has a porosity of at least 40%” is related to the total porosity of the block. The total porosity of the block is at least 40%, such as at least 50% or at least 60%, e.g. from 40 to 80%, e.g. from 60 to 80% or e.g. from 70% to 90%.

The expression “is a geometrical structure shaped to fit at least a part of a degraded alveolar process of the human or animal” implies a structure which fits such an implantation site. This implies that the structure according to the present invention has somewhat of a cubic or rectangular cross section, seen from one surface side (intended bone contacting surface) through the block to the opposite surface side, or vice versa (the intended bone contacting surface of the block according to the present invention is explained below). However, a perfect such cross section is not needed according to the present invention and the cross section may as such have irregular portions and e.g. somewhat rounded ends. For instance an oval cross section is fully possible, however in that case preferably with straight or cut ends and not totally rounded as in a perfect oval shape. However, perfect spheres or cylindrical shapes having a circular cross section, such as the cylindrical start material according to Haugen et al., are not geometrical structures which fit a part of or an entire degraded alveolar process of a human or animal, and are as such not geometrical structures according to the present invention.

Moreover, the expression “shaped to fit at least a part of a degraded alveolar process of the human or animal” implies that the blocks according to the present invention may fit only a part of a degraded alveolar process or an entire such alveolar process. The letter case implies a totally U-shaped (horseshoe shaped) block having a U-bent longitudinal cross section. Blocks intended to fit only part of one side of a degraded alveolar process is of course also possible. Such a structure has also a bent cross section, but not fully bent as in the case of a U-shaped block according to the present invention.

Finally, the expression “continuous pores” is explained below in the description.

### DETAILED DESCRIPTION OF SOME EMBODIMENTS ACCORDING TO THE PRESENT INVENTION

The different porosity characteristics of the block according to the present invention are of great importance, just as the material per se, i.e. having at least a block core consisting of titanium metal or titanium alloy. One such property is continuous pores, another one is the total porosity of the porous block. It is according to the present invention possible to have continuous pores going through the entire block, but it is important to control the surface porosity of at least that surface of the block according to the present invention which is not intended to be in contact with bone, and which is e.g. in contact with soft tissue. This surface can in some embodiments be white. A fully open structure is not desirable, such as the structure according to Haugen et al.

Due to the fact that these porosity properties are related to the possibility of growth and ingrowth of bone tissue when a block according to the present invention is implanted in a human or animal body, it is important that these properties are controlled and kept at the desired level during production of the blocks according to the present invention. According to one specific embodiment of the present invention, the porosity of the porous block is at least 60%, such as e.g. from 60 to 80%. According to yet another embodiment, the porosity of the porous block is at least 80%, such as e.g. from 80 to 90%. A high level of porosity is important for the possible growth and ingrowth of bone cells after implantation, as mentioned, but also for the provision of a material which is easy to handle and process for e.g. a dentist or surgeon before implantation. However, as said, the surface porosity of at least the surface side which is opposite to the intended bone contacting surface and which as such is not intended to be in contact with bone has to be controlled so that connective tissue ingrowth is hindered. It is important to realise that it is possible to control and hinder this phenomena in different ways according to the present invention. This is explained in more detail below.

As said, the shapes and sizes of the porous blocks according to the present invention may vary according to above, but the geometrical shapes are shaped to fit a degraded alveolar process of a human or animal.

According to the present invention, the porous block is a geometrical structure having a width, a height and a length, and having an intended bone contacting surface defined by the width and the length, the intended bone contacting surface intended to be in contact with the bone surface of an implantation site in the maxillofacial area of the human or animal, wherein the average value of the width is in the range of 5-10 mm, such as 5-9, 5-8, 5-7, 5-6, 6-10, 6-9, 6-8, 6-7, 7-10, 7-9, 7-8, 8-10, 8-9 and 9-10 mm, the average value of the height is in the range of 3-10 mm, such as 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 and 9-10 mm, and the average value of the length is in the range of 5-100 mm, such as 5-95, 5-85, 5-75, 5-65, 5-55, 5-45, 5-35, 5-25, 5-15, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90 and 90-100 mm.

The geometrical structure above is defined by average values of the width, length and height, showing that the cross sections of the possible geometrical structures according to the present invention do not have to be perfectly cubic, rectangular or U-shaped, i.e. neither in a straight nor a bent configuration. The geometrical width, length and height may as such be irregular. Moreover, the different sides or surfaces of the blocks according to the present invention may also be irregular. As may be understood from above, the possible configurations according to the present invention have a non-circular cross section, seen from the intended bone contacting surface to the opposite surface side.

As mentioned above, the intended bone contacting surface of the block according to the present invention is the surface intended to be in contact with the degraded alveolar process implantation site. As such, this surface has to be porous and having pores extending through the block so that bone ingrowth is possible through the block with a secure anchoring. As such, some of these pores have to have a pore diameter size of at least 50 μm to ensure bone ingrowth. Other sides of the block according to the present invention, such as especially the opposite surface to the intended bone contacting surface, but also the length sides defined by the length and height, should have limited porosity so that connective tissue ingrowth and other non-bone tissue ingrowth from those sides through the block are depressed. This may be accomplished in different ways according to the present invention. Also the end sides of the blocks according to the present invention, defined by the width and height, may have limited porosity, but this is dependent on the use of the block according to the present invention. In some cases several blocks according to the present invention are intended to be used to form a bridge. In such cases, where the ends of different blocks are linked to each other, these ends may preferably be porous with several pores having a pore diameter size of at least 50 μm. As such bone ingrowth is possible from one block to another in a block bridge to ensure anchoring for the entire bridge of blocks.

Moreover, in some cases there is only a need for a very thin block, such as with a height of the block going down to about 3 mm, and in such cases it is of course preferred to have a thin block due to the fact that otherwise the dentist or surgeon has to remove a lot of material from the block before implantation. The cost of material is also a factor which has importance in such a case. The choice of the specific size and shape of the block according to the present invention is closely related to the use of the block implant and the requirements of the dentist or surgeon going to use the blocks according to the present invention.

As mentioned, the length of the block according to the present invention does not have to be an almost straight length. Due to the fact that the block according to the present invention should fit at least part of a depressed alveolar process of a human or animal, the longer length of the block the more curved it should be. As such, the length of the block according to the present invention does not have to be a straight length but may in fact be a curved length, such as e.g. in the case of the average length being in the range of 15-100 mm. In the case of the average length being 80-100 mm, then the block is U-shaped for sure. Also in a curved configuration, the length is defined by the total distance from one end of the block to the other, following the block all the way from end to end. Also the width and height of the block do not have to be totally straight, but in this case this is normally due to irregular portions and shapes and not curved configuration. As mentioned, this is also the reason why the blocks according to the present invention have to be defined by average values.

According to one specific embodiment of the present invention, the average value of the width is in the range of 5-7 mm, such as 5-6 and 6-7 mm, the average value of the height is in the range of 3-5 mm, such as 3-4 and 4-5 mm, and the average value of the length is in the range of 5-15 mm, such as e.g. 5-13, 5-11, 5-9, 5-7, 7-15, 7-13, 7-11, 7-9, 8-15, 9-15, 9-13, 9-11, 10-15, 11-15, 11-13 and 13-15 mm.

The intended use of the blocks according to the present invention is as implants for growth and ingrowth of bone tissue/bone cells in the maxillofacial area of a human or animal. To be able to grow bone cells through or into the pores, the pores should, as mentioned, have openings having diameters of at least 50 μm, where some pores may have diameters as large as up to 600 pm or even larger. According to one specific embodiment of the present invention, at least 50% of the continuous pores have pore opening diameters of at least 50 μm.

As will be explained in more detail below, it is important to make sure that connective tissue ingrowth is not favoured over bone tissue ingrowth. Therefore, when the block according to the present invention has surfaces or sides with pores which surfaces or sides are not intended to be in contact with bone, these surfaces or sides have to be protected so that substantially no connective tissue ingrowth is possible. This is possible to accomplish by e.g. a protective membrane. However, it is also possible according to the present invention to provide such surfaces or sides without porosity. The intended bone contacting surface, i.e. the surface intended to be in contact with the degraded alveolar process, should, however, have surface porosity and of course with a large amount of pores having a pore diameter of at least 50 μm, so as to promote bone tissue ingrowth through the pores.

The pore openings are of course not perfectly round or circular in most cases, and the expression “pore diameter” should in this sense be interpreted as a mean value for all lengths going from one side of the pore opening to the other side, through a geometrical centre, of the specific pore. It is also important to realise that some pores and normally a lot of the pores of the block according to the present invention are not continuous. However, these non-continuous pores increase the total porosity and are also important when it comes to the structural shape, such as creating a possible irregularity on the surface of the block. Therefore, according to one specific embodiment of the invention, the porous block has an irregular surface, which in fact could be said to be a result of small and large pores in the surface of the block, continuous as well as non-continuous ones.

There exist different possible maxillofacial uses for blocks according to the present invention. According to one specific embodiment of the present invention, the porous block is shaped to fit a part of or an entire degraded alveolar process of the maxilla of the human or animal. Also in this case, the intended bone contacting surface of the block according to the present invention is the intended site to be in contact with the implantation site.

As mentioned, blocks according to the present invention may be used together with other blocks to form a bridge. According to one specific embodiment of the present invention, at least one end of the two ends in a longitudinal direction of the porous block is cut and shaped to fit another cut and shaped end of a porous block. The surfaces of the ends of the block according to the present invention are defined by the width and height. Moreover, as said, the surfaces of these ends may have surface porosity due to the fact that bone ingrowth may grow from one block and in to the other block. This is, however, of course an optional feature according to the present invention.

The blocks according to the present invention may e.g. be implanted together with at least one titanium screw and/or at least one other type of fastener for fixating the porous block into living bone of a degraded alveolar process of a human or animal body. In these cases, the blocks according to the present invention are provided with predrilled holes that suit together with the screws or other type of fasteners. Therefore, according to one specific embodiment, at least one screw hole is provided through the porous block. Such screw holes are provided from the intended bone contacting surface through the entire porous block and out on the other opposite surface side.

As mentioned, it is important to decrease the risk of connective tissue ingrowth while this may depress bone tissue ingrowth. Therefore, surface porosity of the block according to the present invention should preferably only exist where only bone tissue ingrowth is possible. Other surfaces should preferably not be porous, at least not have continuous pores. Therefore, according to one specific embodiment of the present invention, only the intended bone contacting surface of the porous block and optionally the two ends in a longitudinal direction of the porous block are porous in the surface.

It is according to the present invention possible to achieve the bone ingrowth properties of the porous block in different ways. The common feature of the porous blocks according to the present invention, according to some specific embodiments, is the non-porous surfaces of surfaces where there is an evident risk for soft tissue ingrowth after implantation. This feature may be accomplished or provided in different ways. Therefore, according to one specific embodiment of the present invention, there is provided a porous block wherein at least one portion of a surface of the porous block is protected from possible soft tissue ingrowth after implantation by means of being non-porous or by means of an outer protecting membrane or membrane-like structure.

According to one specific embodiment, said at least one portion of a surface, or one or more sides of the porous block, is protected by a non-porous laminated surface layer.

According to another embodiment, said at least one portion of a surface of the porous block is protected by being provided with an outer skin. As is discussed below, an outer skin covering the block according to the present invention may be provided during the production, such as by a gel forming process. The covering outer skin may be oxidised and finally one or more sides of the block are cut or fractured so that porous sides are exposed. As such, a block according to the present invention may have non-porous sides which are oxidised as well as sides which are porous and consist of titanium oxide, titanium metal or titanium alloy. However, the block according to the present invention is not a 100% titanium oxide block.

According to another embodiment of the present invention, there is provided a protective membrane or film in a kit, which membrane or film protects porous surfaces of the block according to the present invention. As mentioned above, in another embodiment the non-permeable surface is created by laminating a membrane or a surface layer film onto one or more surfaces of the block. According to yet another embodiment of the present invention, such a membrane or film is applied directly during production. According to yet one other embodiment of the present invention, the production is made so that a non-surface porous block is obtained directly during the production thereof. This is obtained with a foaming technique. In such a case the entire produced block is porous inside but has a surface all over which is non-porous. In such a case, the intended bone contacting side is then cut so that this side becomes porous.

The latter case, with a process according to the present invention giving a porous block having a surface which is non-porous, is possible to achieve by different foaming techniques, such as e.g. gel foaming. In the gel forming process all external surfaces including those in contact with the mould or atmosphere are covered with a non-permeable “skin”. A surface with open porosity is created by exposing the internal porosity by cutting or fracturing the block. The intended bone contacting surface is thus cut so that the continuous pores through the block are uncovered on the intended side. In such a case, the shape of the block must be decided before the production so that only minor cutting is needed afterwards. Otherwise, if much cutting is needed, the non-porous surfaces would be lost.

However, it is also possible to encapsulate porous sides of a block according to the present invention so that a porous surface is protected from connective tissue ingrowth. Such a configuration should according to the present invention also be interpreted as a non-porous surface. For instance, different membranes are possible to use to encapsulate porous surfaces which preferably should not be porous. A block according to the present invention which has several porous surfaces with continuous porosity through the entire block may be provided together with such a membrane in a kit according to the present invention. Moreover, as is explained above, a non-permeable surface layer film which is surface treated to be whitish may also be laminated to one or more sides of the block.

To make the expression “continuous pores” clearer, this implies pores going through the entire block. In relation to the present invention it is important to understand that such continuous pores exist and are vital for bone ingrowth, but that surfaces where there is an evident risk for soft tissue ingrowth after implantation are protected and as such are non-porous. However, inside of the block these continuous pores go through the entire block from one side of the block to the other. Moreover, these continuous pores may be interconnected with other pores also, i.e. creating a network of pore channels being interconnected to each other, so that one continuous pore system is provided inside.

As is disclosed above, according to one embodiment, there is provided a kit. This kit may comprise a porous block according to present invention and a biocompatible membrane being of natural or synthetic origin. The biocompatible membrane is intended for use as a sealing on top of the porous block when implanted, such as to prevent the ingrowth of a wrong type of tissue that is not bone tissue, such as connective tissue. Moreover, the membrane may function as a sealing that also keeps the porous block in the right place, such as when screws or other fasteners are not used to anchor the block per se. The biocompatible membrane according to the invention may be of synthetic origin, e.g. materials comprising polymers, Teflon® (PTFE), silicone, polyethylene, polylactic acid (degradable), polyglycolic acid (degradable) or metals, or combinations thereof. Examples of suitable metals are titanium, tantalum, zirconium or alloys thereof. Ceramics are also possible, such as oxides of titanium, tantalum, zirconium or aluminium. The biocompatible membrane may also be of natural origin, such as natural polymers, collagen or silk. Moreover, the biocompatible membrane according to the present invention may be resorbable or permanent (non-resorbable). Other possible membrane materials are different waxes or e.g. gypsum. Such materials may fill pores of porous sides of a block according to the present invention which sides should not be held porous after implantation.

There exist known ways for producing different types of titanium sponges, which could be used as the start material for the production of an specifically shaped titanium and/or titanium alloy implant block according to the present invention. Such processes are e.g. the well-known Hunter process or Kroll process. Other existing techniques suitable to use when producing the start material are e.g. a direct foaming technique as gel casting, or other wet processing methods as a replication technique and a rapid prototyping technique. Sintering is also a process often combined with the technique above to produce porous titanium or titanium alloy sponges. From such a start material, it is according to the present invention possible to produce an implant block.

According to one embodiment of the present invention, the process for producing a porous block according to the present invention comprises use of the techniques above and then reshaping the achieved start materials for producing blocks being geometrical structures shaped to fit at least a part of a degraded alveolar process of the human or animal. Such geometrical shapes may also be pre-shaped to fit the individual patient's needs by using CAD CAM techniques, which is mentioned above.

As is mentioned above, it may be of interest to produce a porous block comprising titanium metal and having an outer layer of titanium dioxide. This is possible according to the present invention by a process comprising the steps of:
a semi-oxidation of an optionally pre-shaped porous block comprising titanium metal and optionally having an irregular surface, in a temperature of at least 300° C. in an oxygen containing atmosphere during enough time for achieving a semi-oxidation to a porous titanium block having a titanium dioxide layer on its surface; and
optionally shaping the achieved porous titanium block to a desired shape with appropriate means.

With reference to the present invention, the expression “semi-oxidation” implies a partial oxidation and not a full-oxidation. It is possible to perform the semi-oxidation in different ways. Using a comparatively low temperature, such as 300° C.-600° C. implies that the oxidation will occur slower. To make sure that the oxidation is not made completely to a 100% titanium dioxide block it is important to control the temperature and the oxygen level and also the oxidation time. Moreover, a higher temperature and controlled oxygen level are preferred for the oxidation to keep the oxidation time within a reasonable limit and have a reaction which is not dangerous. Therefore, according to another embodiment, the semi-oxidation is performed in a temperature of at least 800° C. Moreover, a semi-oxidation made by anodic oxidation, or other similar surface modification, is also possible.

Moreover, as mentioned above, if the start material is made by a gel forming process all external surfaces including those in contact with the mould or atmosphere are in most instances covered with a non-permeable “skin”. The surface may then be treated according to above by a semi-oxidation or a surface oxidation so that the surface is oxidised. Thereafter, one or more sides of the block are cut or fractured so that the internal porous structure of the block is uncovered. Dependent on the magnitude of the oxidation, the uncovered “new” porous surfaces consist of titanium oxide, or in fact titanium metal or titanium alloy. As mentioned above, preferably the uncovered surfaces consist of the titanium metal or titanium alloy. According to this embodiment of the present invention, the block has one or more non-porous sides being whitish in view of the oxidation and also one or more porous sides which consist of titanium metal or titanium alloy.

The above expression “optionally pre-shaped” implies that it is possible that the start material, i.e. the titanium sponge, at the gel casting stage or other suitable stages of wet processing methods, etc., is shaped in a desired shape already at these stages. Another possibility is to shape by grinding or milling of the desired blocks comprising titanium metal and optionally having an irregular surface, in advance, which blocks subsequently according to the present invention may be semi-oxidised. This can be done at any temperature but the preferred condition is at a temperature of at least 800° C. in air during enough time for achieving a semi-oxidation to a porous titanium block according to the present invention. The temperature may be above 800° C. and e.g. up to 1000° C. and above, and this is regulated with the retention time of the oxidation, so that a semi-oxidation is achieved.

Subsequently to the possible semi-oxidation according to this specific embodiment of the present invention, the achieved titanium block having a surface layer of titanium dioxide may be further shaped to a desired shape, such as by e.g. crushing, grinding, milling and e.g. filing, which is discussed above. For example, a cutting step disclosed below is such a further shaping step.

As is disclosed above, the production according to the present invention may also be directed to the provision of a porous block having only directed or predetermined surface porosity, which gives a block having surface porosity on only intended sites. Therefore, the process according to the present invention may also comprise a foaming step for the production of a porous block having non-porous surfaces. The intended site having surface porosity, i.e. the surface intended to be in contact with a bone implantation site, thereafter has to be provided, if not already made during the foaming step. Therefore, the process may additionally comprise a cutting step for the production of a porous block having at least one porous surface intended to be in contact with a bone surface after implantation. Such a cutting step will uncover one side of the porous block so that the continuous pores and the pore network of that side are unprotected, ensuring possible bone ingrowth from that side. This is further explained above, such as in the case of a block having one or more non-porous surfaces of titanium dioxide and one or more uncovered sides of titanium metal or titanium alloy.

The process according to the present invention is different from the polymer sponge method disclosed by Haugen et al, which polymer sponge method gives a fully open structure and a cylindrical shape. According to the present invention, the process ensures both intended shape and surface porosity.

According to the present invention there is further provided the use of a porous block or a kit according to the present invention, together with at least one titanium screw and/or at least one other type of fastener for fixating the porous block into living bone in the maxillofacial area of a human or animal subject. Specific examples are for implantation in/at the lower jawbone (mandible) or upper jawbone (maxilla) of the human or animal subject. The blocks according to the present invention may in this type of applications be implanted for augmentation, e.g. of the upper jawbone, and/or for bone growth and ingrowth through the continuous and interconnected pores of the porous block, i.e. as a builder of bone at the intended site.

The block material according to the present invention may also be used for applications other than dental, such as e.g. for orthopaedic implantation at the site for a hip joint replacement or as an augmentation of such a replacement or a prosthesis therefore. However, this is dependent on the composition of the block. If the block core e.g. is made of titanium alloy, it is possible to achieve very strong blocks which may bear high mechanical loads. This is a clear difference in comparison to a block entire being made of titanium dioxide. Such a titanium dioxide block has limited use in relation to implantation at sites exposed to extreme level of mechanical loads exerted by e.g. direct contact with a major load bearing prosthesis. Nevertheless, in all situations when the titanium metal or titanium alloy block is only in contact with bone, the strength of the block is sufficient as it is stronger than the bone it is in contact with.

A dentist or surgeon may shape the porous block to a desired shape before implantation. For example, when a dentist is about to implant a block according to the present invention, for e.g. augmentation of an upper jawbone and growth and ingrowth of bone tissue, it may be of importance to shape the block in the appropriate shape in accordance with the existing upper jaw of the patient being treated. This may e.g. be made by filing or in another suitable way.

Moreover, as mentioned above, it is possible to apply the porous block(s) with a membrane to further improve the chances of bone ingrowth into the block(s). As mentioned, the porous blocks according to the present invention, may be implanted together with at least one titanium screw and/or at least one other type of fastener for fixating the porous block into living bone of the human or animal body at the intended site. This is also valid for the sealing membrane, i.e. the membrane may be fixated with a fastener in the same way as the porous block itself, where such a fastener, like a titanium screw, is driven through the membrane, the porous block and into the living bone, to fasten the entire kit.

### Conclusions

The block implants according to the present invention have several advantages in comparison to existing implants. The blocks according to the present invention, comprising titanium metal and/or a titanium alloy, are easy to reshape, process and handle for a dentist or surgeon about to implant such a block. This is not the case with titanium implants or similar implants of other metals available on the market today. Furthermore, as the implant blocks according to the present invention have advantageous properties with reference to shape, size and porosity, they provide good bone growth and ingrowth after implantation. Due to the directed surface porosity of the blocks according to the present invention, it is possible to optimise bone ingrowth in maxillofacial implantation applications, such as by depressing soft tissue ingrowth. Furthermore, as the blocks according to the present invention comprise titanium metal or a titanium alloy they are easier to handle than a block consisting of 100% titanium dioxide. This is due to the fact that they are easier to drill into and reshape in comparison to a block being made of titanium dioxide. Furthermore, they sustain higher mechanical loads than a block entirely being made of titanium dioxide. Moreover, as the implant blocks according to the present invention may be provided with a titanium oxide layer or a surface treated non-permeable surface layer film, they may have a whitish appearance and as such being very suitable to use in dental applications as they give a favourable aesthetic appearance.

1. Porous block for implantation in the maxillofacial area of a human or animal, wherein the porous block:
comprises titanium metal and/or a titanium alloy;
has a porosity of at least 40%; and
is a geometrical structure shaped to fit at least a part of a degraded alveolar process of the human or animal; and
has an intended bone contacting surface intended to be in contact with the bone surface of an implantation site in the maxillofacial area of the human or animal, wherein said bone contacting surface has pores extending through the porous block and wherein at least some of these pores have a pore diameter size of at least 50 μm to ensure bone ingrowth; wherein
the porous block is a geometrical structure having a width, a height and a length, said intended bone contacting surface being defined by the width and the length, wherein the average value of the width is in the range of 5-10 mm, the average value of the height is in the range of 3-10 mm and the average value of the length is in the range of 5-100 mm; and wherein
the porous block comprises an outer layer of titanium oxide on one or more surface sides or surface portions of the porous block.

2. Porous block according to claim 1, wherein the porous block comprises titanium metal and/or titanium alloy in an amount of at least 5 wt % in total.

3. Porous block according claim 1, wherein the porous block comprises titanium metal and/or titanium alloy in an amount of at least 25 wt % in total.

4. Porous block according to claim 1, wherein the porous block comprises titanium metal and/or titanium alloy in an amount of at least 50 wt % in total.

5. Porous block according to claim 1, wherein the porous block comprises titanium metal and/or titanium alloy in an amount of at least 75 wt % in total.

6. Porous block according to claim 1, wherein the porous block comprises titanium metal and/or titanium alloy in an amount of at least 95 wt % in total.

7. Porous block according to claim 1, wherein the porous block comprises titanium metal and/or titanium alloy in an amount of at least 99 wt % in total.

8. Porous block according to claim 1, wherein the outer layer of titanium oxide on one or more surface sides or surface portions of the porous block is applied by a performed oxidation.

9. Porous block according to claim 1, wherein the titanium oxide is titanium dioxide.

10. Porous block according to claim 1, wherein the porosity of the porous block is at least 60%.

11. Porous block according to claim 1, wherein the porosity of the porous block is at least 80%.

12. Porous block according to claim 1, wherein at least one portion of a surface of the porous block is protected from possible soft tissue ingrowth after implantation by means of being non-porous or by means of an outer protecting membrane or membrane-like structure.

13. Porous block according to claim 12, wherein at least one portion of a surface of the porous block is protected by a non-porous laminated surface layer.

14. Porous block according to claim 12, wherein at least one portion of a surface of the porous block is protected by being provided with an outer skin.

15. Porous block according to claim 14, wherein the outer skin is oxidized.

16. Porous block according to claim 1, wherein the average value of the width is in the range of 5-7 mm, the average value of the height is in the range of 3-5 mm and the average value of the length is in the range of 5-15 mm.

17. Porous block according to claim 1, wherein the porous block is shaped to fit a part of or an entire degraded alveolar process of the maxilla of the human or animal.

18. Porous block according to claim 1, wherein at least one end of the two ends in a longitudinal direction of the porous block is cut and shaped to fit another cut and shaped end of a porous block.

19. Porous block according to claim 1, wherein at least one screw hole is provided through the porous block.

20. Porous block according to claim 1, wherein only the intended bone contacting surface of the porous block and optionally the two ends in a longitudinal direction of the porous block are porous in the surface.

21. Kit comprising a porous block according to claim 1 and a biocompatible membrane being of natural or synthetic origin.

22. Kit according to claim 21, wherein the biocompatible membrane is resorbable or permanent.

23. Use of a porous block according to claim 1 or a kit according to claim 21 or 22, for implantation in the maxillofacial area of a human or animal.

24. Use of a porous block according to claim 1 or a kit according to claim 21 or 22, together with at least one titanium screw and/or at least one other type of fastener for fixating the porous block into living bone in the maxillofacial area of a human or animal subject.
